(19) United States
(12) Patent Application Publication
(10) Pub. No.: US 2026/0347400 A1
(43) Pub. Date: Nov. 5, 2026

(54) ANTIMICROBIAL PEPTIDE FOR MAINTAINING BALANCE OF ORAL FLORA AND METHOD FOR PREVENTION OR TREATMENT OF ORAL DISEASE USING THE SAME

(71) Applicant: Metanovas Biotech Inc., San Francisco, CA (US)

(72) Inventors: Lun YU, San Jose, CA (US); Meijie WANG, Santa Clara, CA (US); Heng LUO, Brooklyn, NY (US)

(21) Appl. No.: 19/193,918

(22) Filed: Apr. 29, 2025

(51) Int. Cl. C07K 7/08; A61K 38/00

(52) U.S. Cl. CPC C07K 7/08; A61K 38/00

(57) ABSTRACT

An antimicrobial peptide for maintaining the balance of oral flora including an amino acid sequence shown in SEQ ID NOs: 1, 2, 3, 4, or a variant thereof. The antimicrobial peptide exhibits distinct antimicrobial activity against oral flora. According to specific embodiments of the disclosure, the antimicrobial peptide demonstrates good antimicrobial activity against key harmful oral bacteria, including Streptococcus heteroclitus, Porphyromonas gingivalis and Helicobacter pylori, and can be used for maintaining the balance of oral flora.

### INCORPORATION-BY-REFERENCE OF SEQUENCE LISTING

This application comprises a sequence listing, which has been submitted electronically in XML file and is incorporated herein by reference in its entirety. The XML file, created on Apr. 7, 2025, is named SZWK-00201-UUS.xml, and is 18,979 bytes in size.

### BACKGROUND

The disclosure relates to the field of antimicrobial peptides, and more particularly, to an antimicrobial peptide for maintaining the balance of oral flora and a method for prevention or treatment of an oral disease using the same.

The oral cavities of adults harbor an enormous population of bacteria, ranking first among all body sites in terms of both quantity and species diversity. Under normal circumstances, these bacteria maintain a dynamic balance through symbiosis, competition, and antagonism. The equilibrium among microbial communities and between the microbes and the host is generally harmless and non-pathogenic, constituting what is known as the oral normal microbiota, which plays a positive role in maintaining oral health. However, endogenous infections may occur when this microbial balance is disrupted, when microbial virulence factors are enhanced, or when the host's oral defense mechanisms become compromised.

Antimicrobial peptides (AMPs) are evolutionarily conserved immune-active molecules produced by organisms as part of their environmental adaptation. They serve as crucial mediators of innate immunity. Functioning as “natural antimicrobial agents”, AMPs play a pivotal role in host defense against pathogenic invasions. Beyond directly killing pathogens, AMPs more frequently act as immunomodulatory effectors that initiate and regulate the host's immune defense system.

Despite their significance, natural AMPs face limitations in practical applications due to high production costs, low antimicrobial activity, and poor stability. With advancing research on the properties, structures, and mechanisms of existing natural AMPs, increasing efforts are being directed toward the development of synthetic antimicrobial peptides. Currently, research on AMPs for maintaining oral microbial balance remains relatively limited.

### SUMMARY

To address the aforesaid problems, the disclosure provides an antimicrobial peptide for maintaining the balance of oral flora.

In another aspect, the disclosure further provides a method for prevention or treatment of an oral disease caused by dysbiosis of oral microbiota.

The antimicrobial peptide for maintaining the balance of oral flora of the disclosure is selected from one of the following polypeptides (1)-(7):
(1) a polypeptide comprising an amino acid sequence shown in SEQ ID NO: 1, or a polypeptide consisting of amino acid residues of SEQ ID NO: 1;
(2) a polypeptide comprising an amino acid sequence shown in SEQ ID NO: 2, or a polypeptide consisting of amino acid residues of SEQ ID NO: 2;
(3) a polypeptide comprising an amino acid sequence shown in SEQ ID NO: 3, or a polypeptide consisting of amino acid residues of SEQ ID NO: 3;
(4) a polypeptide comprising an amino acid sequence shown in SEQ ID NO: 4, or a polypeptide consisting of amino acid residues of SEQ ID NO: 4;
(5) a polypeptide resulting from substitution, deletion, and/or addition of one or more amino acid residues of the polypeptide in (1), (2), (3) or (4);
(6) a polypeptide obtained by modifying a N-terminus and/or a C-terminus of the polypeptide in (1), (2), (3) or (4); and
(7) a polypeptide having at least 85%, 90%, 95%, 98%, or 99% sequence identity to the amino acid sequence of the polypeptide in (1), (2), (3) or (4).

In a class of this embodiment, the antimicrobial peptide is antimicrobial peptide AMP8, antimicrobial peptide AMP25, antimicrobial peptide AMP29 or antimicrobial peptide AMP31.

In a class of this embodiment, the amino acid sequence of the antimicrobial peptide AMP8 is shown in SEQ ID NO: 1, the amino acid sequence of the antimicrobial peptide AMP25 is shown in SEQ ID NO: 2, the amino acid sequence of the antimicrobial peptide AMP29 is shown in SEQ ID NO: 3, and the amino acid sequence of the antimicrobial peptide AMP31 is shown in SEQ ID NO: 4.

The disclosure also provides a method for preparing the aforementioned antimicrobial peptide for maintaining the balance of oral flora. Optionally, this method employs a standard solid-phase synthesis technique.

The disclosure also provides an expression gene configured to express the aforementioned antimicrobial peptide for maintaining the balance of oral flora.

In a class of this embodiment, the expression gene is configured to express the antimicrobial peptide AMP8, the antimicrobial peptide AMP25, the antimicrobial peptide AMP29, or the antimicrobial peptide AMP31.

In a class of this embodiment, the amino acid sequence of the antimicrobial peptide AMP8 is shown in SEQ ID NO: 1, the amino acid sequence of the antimicrobial peptide AMP25 is shown in SEQ ID NO: 2, the amino acid sequence of the antimicrobial peptide AMP29 is shown in SEQ ID NO: 3, and the amino acid sequence of the antimicrobial peptide AMP31 is shown in SEQ ID NO: 4.

The disclosure also provides a method for prevention or treatment of an oral disease caused by dysbiosis of oral microbiota, the method comprising: administering to a patient in need thereof a product comprising a therapeutically effective amount of the aforementioned antimicrobial peptide.

In a class of this embodiment, the oral disease is selected from the group consisting of pulpitis, apical periodontitis, gingivitis, periodontitis, stomatitis, and dental caries.

In a class of this embodiment, the product is selected from the group consisting of toothpaste, mouthwash, effervescent mouthwash tablets, oral care products, and topical medicaments.

The antimicrobial peptide of the disclosure for maintaining the balance of oral flora is synthesized artificially after virtual screening, and exhibits distinct antimicrobial activity against oral flora. According to specific embodiments of the disclosure, the antimicrobial peptide demonstrates good antimicrobial activity against key harmful oral bacteria, including Streptococcus heteroclitus, Porphyromonas gingivalis and Helicobacter pylori, and can be used for maintaining the balance of oral flora.

### BRIEF DESCRIPTION OF THE DRAWINGS

FIG. 1 shows a high-performance liquid chromatography (HPLC) result of the antimicrobial peptide AMP29 prepared in Example 3;

FIG. 2 shows a mass spectrometry (MS) result of the antimicrobial peptide AMP29 prepared in Example 3;

FIG. 3 shows a bacteriostatic test result of the antimicrobial peptide AMP8, antimicrobial peptide AMP25, antimicrobial peptide AMP29, and antimicrobial peptide AMP31 against Streptococcus mutans in Example 6;

FIG. 4A shows a bacteriostatic test result of the antimicrobial peptides AMP29 with a concentration of 5 μg/mL against Porphyromonas gingivalis in Example 7;

FIG. 4B shows a bacteriostatic test result of the antimicrobial peptides AMP29 with a concentration of 10 μg/mL against Porphyromonas gingivalis in Example 7;

FIG. 5A shows a bacteriostatic test result of the antimicrobial peptides AMP29 and AMP31 with a concentration of 5 μg/mL against Helicobacter Pylori in Example 8;

FIG. 5B shows a bacteriostatic test result of the antimicrobial peptides AMP29 and AMP31 with a concentration of 10 μg/mL against Helicobacter Pylori in Example 8;

FIG. 6 shows a bacteriostatic test result of the antimicrobial peptide AMP8, the antimicrobial peptide AMP25, the antimicrobial peptide AMP29, and the antimicrobial peptide AMP31 against Candida albicans in Example 9;

FIG. 7 shows an inhibition result of the antimicrobial peptides AMP8, AMP25, AMP29, and AMP31 against LPS-induced inflammation in mouse macrophage RAW264.7 cells in Example 10;

FIG. 8 shows a bacteriostatic test result of the variants of the antimicrobial peptide AMP29 against Streptococcus mutans; and

FIG. 9 shows a bacteriostatic test result of the variants of the antimicrobial peptide AMP29 against Staphylococcus aureus.

### DETAILED DESCRIPTION

To further illustrate the disclosure, embodiments detailing an antimicrobial peptide for maintaining the balance of oral flora and method for prevention or treatment of an oral disease using the same are described below. It should be noted that the following embodiments are intended to describe and not to limit the disclosure.

With the development of artificial intelligence, AI model-based approaches have been applied to design antimicrobial peptides (AMPs). AI model-based ab initio antimicrobial peptide design employs two types of models: (1) generative models that generate new peptide sequences, and (2) predictive models that forecast the biological activities and properties of the peptides based on the peptide sequences as inputs.

To obtain antimicrobial peptides for maintaining oral flora balance, the disclosure employed a deep learning process involving a series of deep learning (DL) models. This process utilized generators and classifiers based on transfer learning and pre-trained proteins for embedding and training. Ultimately, four antimicrobial peptides (AMP8, AMP25, AMP29, and AMP31) were obtained and tested in vitro. The antimicrobial activities of these four peptides were validated, demonstrating their potential for maintaining oral flora balance.

Thus, the disclosure provides an antimicrobial peptide for the maintenance of oral flora balance, and the antimicrobial peptide is selected from one of the following polypeptides (1)-(7):
(1) a polypeptide comprising an amino acid sequence shown in SEQ ID NO: 1, or a polypeptide consisting of amino acid residues of SEQ ID NO: 1;
(2) a polypeptide comprising an amino acid sequence shown in SEQ ID NO: 2, or a polypeptide consisting of amino acid residues of SEQ ID NO: 2;
(3) a polypeptide comprising an amino acid sequence shown in SEQ ID NO: 3, or a polypeptide consisting of amino acid residues of SEQ ID NO: 3;
(4) a polypeptide comprising an amino acid sequence shown in SEQ ID NO: 4, or a polypeptide consisting of amino acid residues of SEQ ID NO: 4;
(5) a polypeptide resulting from substitution, deletion, and/or addition of one or more amino acid residues of the polypeptide in (1), (2), (3) or (4);
(6) a polypeptide obtained by modifying a N-terminus and/or a C-terminus of the polypeptide in (1), (2), (3) or (4); and
(7) a polypeptide having at least 85%, 90%, 95%, 98%, or 99% sequence identity to the amino acid sequence of the polypeptide in (1), (2), (3) or (4).

SEQ ID NO: 1:
KLVKRVLKKLRKLF.
SEQ ID NO: 2:
AKRIVKLIKNFFRKL.
SEQ ID NO: 3:
KKIFKRIVKIIKRLL.
SEQ ID NO: 4:
KILGKLLKWASKIW.

The antimicrobial peptide of the disclosure for maintaining the balance of oral flora is synthesized artificially after virtual screening, and exhibits distinct antimicrobial activity against oral flora. According to specific embodiments of the disclosure, the antimicrobial peptide demonstrates good antimicrobial activity against key harmful oral bacteria, including Streptococcus heteroclitus, Porphyromonas gingivalis and Helicobacter pylori, and can be used for maintaining the balance of oral flora.

The antimicrobial peptide of the disclosure for maintaining the balance of oral flora may be a polypeptide obtained by modification of the N-terminus and/or C-terminus, for example, the N-terminal end of the polypeptide is acetylated.

Preferably, in certain embodiments, the antimicrobial peptide is antimicrobial peptide AMP8, antimicrobial peptide AMP25, antimicrobial peptide AMP29 or antimicrobial peptide AMP31.

Preferably, in certain embodiments, the amino acid sequence of the antimicrobial peptide AMP8 is shown in SEQ ID NO: 1; the amino acid sequence of the antimicrobial peptide AMP25 is shown in SEQ ID NO: 2; the amino acid sequence of the antimicrobial peptide AMP29 is shown in SEQ ID NO: 3; and the amino acid sequence of the antimicrobial peptide AMP31 is shown in SEQ ID NO: 4.

The disclosure also provides a method for preparing the above-described antimicrobial peptide for maintaining the balance of oral flora.

In the disclosure, the antimicrobial peptide for maintaining the balance of oral flora is prepared using a standard solid-phase synthesis method.

In certain embodiments, the above-described antimicrobial peptide for maintaining the balance of oral flora can also be prepared by a biosynthetic method.

Furthermore, the disclosure also provides an expression gene configured to express the aforementioned antimicrobial peptide for maintaining the balance of oral flora.

Preferably, in certain embodiments, the expression gene is configured to express the antimicrobial peptide AMP8, the antimicrobial peptide AMP25, the antimicrobial peptide AMP29, or the antimicrobial peptide AMP31.

Preferably, in certain embodiments, the amino acid sequence of the antimicrobial peptide AMP8 is shown in SEQ ID NO: 1; the amino acid sequence of the antimicrobial peptide AMP25 is shown in SEQ ID NO: 2; the amino acid sequence of the antimicrobial peptide AMP29 is shown in SEQ ID NO: 3; and the amino acid sequence of the antimicrobial peptide AMP31 is shown in SEQ ID NO: 4.

The disclosure also provides a method for prevention or treatment of an oral disease caused by dysbiosis of oral microbiota, the method comprising: administering to a patient in need thereof a product comprising a therapeutically effective amount of the aforementioned antimicrobial peptide.

Preferably, in certain embodiments, the oral disease is selected from the group consisting of pulpitis, apical periodontitis, gingivitis, periodontitis, stomatitis, and dental caries.

Preferably, in certain embodiments, the product is selected from the group consisting of toothpaste, mouthwash, effervescent mouthwash tablets, oral care products, and topical medicaments.

Preferably, in certain embodiments, the oral care product is an oral dissolving film or an oral rinse.

Unless otherwise stated, the experimental methods used in the following examples are conventional, and the materials, reagents, and the like used in the examples are commercially available.

In certain examples, Streptococcus mutans ATCC 35668 was purchased from Wuhan Huizao Biotechnology Co., Ltd. (Item No.: HZB360306); Porphyromonas gingivalis ATCC 33277 was purchased from Wuhan Huizao Biotechnology Co., Ltd. (Item No.: HZB354945); Helicobacter pylori ATCC 43504 was purchased from Wuhan Huizao Biotechnology Co., Ltd. (Item No.: HZB369015); Candida albicans ATCC 14053 was purchased from China Center of Industrial Culture Collection (Item No.: 0332P); mouse macrophage RAW264.7 cells were purchased from Wuhan Shangen Biotechnology Co., Ltd. (Item No.: SNL-112). Staphylococcus aureus ATCC: 25923 was purchased from Ningbo Mingzhou Biotechnology Co., Ltd. (Item No. B81854 (A)).

### Example 1

The antimicrobial peptide AMP8, with an amino acid sequence shown in SEQ ID NO: 1, was prepared by GenScript Biotech Corporation using a solid-phase synthesis method.

### Example 2

The antimicrobial peptide AMP25, with an amino acid sequence shown in SEQ ID NO: 2, was prepared by GenScript Biotech Corporation using a solid-phase synthesis method.

### Example 3

The antimicrobial peptide AMP29, with an amino acid sequence shown in SEQ ID NO: 3, was prepared by GenScript Biotech Corporation using a solid-phase synthesis method.

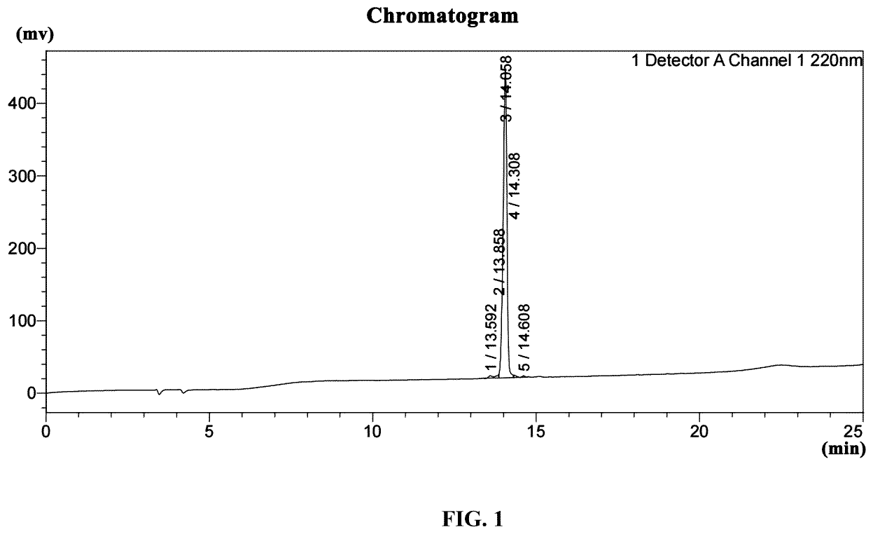

The synthesized antimicrobial peptide AMP29 was subjected to high performance liquid chromatography (HPLC) and the results were shown in Table 1 and FIG. 1.

The operation parameters are as follows:
Sample Name: AMP29
Sample ID: C050Q707G0-1
Time Processed: 11:14:30
Date Processed (Month-Day-Year): 12/06/2024
Pump A: 0.065% trifluoroacetic acid in 100% water (v/v)
Pump B: 0.05% trifluoroacetic acid in 100% acetonitrile (v/v)
Total Flow: 1 mL/min
Wavelength: 220 nm

### <<LC Time Program>>

| Time | Module | Command | Value |
|---|---|---|---|
| 0.01 | Pumps | B.Conc | 5 |
| 25.00 | Pumps | B.Conc | 65 |
| 25.01 | Pumps | B.Conc | 95 |
| 27.00 | Pumps | B.Conc | 95 |
| 27.01 | Pumps | B.Conc | 5 |
| 35.00 | Pumps | B.Conc | 5 |
| 35.01 | Controller | Stop | |

TABLE 1

| Peak# | Ret. Time | Area | Height | Area % |
|---|---|---|---|---|
| 1 | 13.592 | 26692 | 3331 | 0.759 |
| 2 | 13.858 | 26204 | 5101 | 0.745 |
| 3 | 14.058 | 3432153 | 425941 | 97.614 |
| 4 | 14.308 | 18981 | 3339 | 0.540 |
| 5 | 14.608 | 12022 | 1998 | 0.342 |
| Total | | 3516052 | 439711 | 100.000 |

Detector A Channel 1 220 nm.

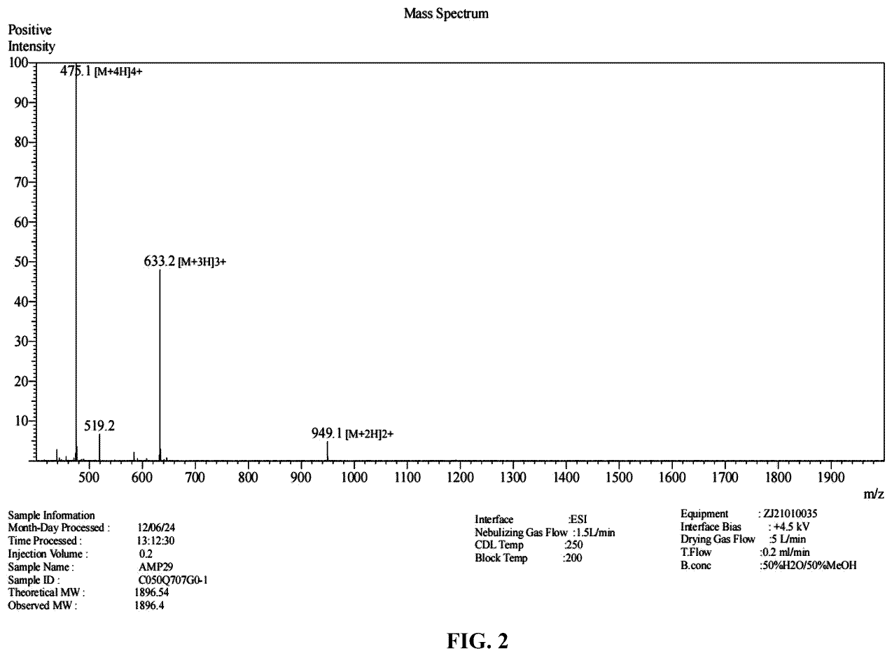

The synthesized antimicrobial peptide AMP29 was subjected to mass spectrometry (mass spectrum, MS) and the results are shown in FIG. 2.

The results from Table 1, FIG. 1 and FIG. 2 indicate that the synthesized antimicrobial peptide AMP29 meets the requirements.

### Example 4

The antimicrobial peptide AMP31, with an amino acid sequence shown in SEQ ID NO: 4, was prepared by GenScript Biotech Corporation using a solid-phase synthesis method.

### Example 5

The variants V1-V15 of the antimicrobial peptide AMP29, with the following amino acid sequences, were prepared by GenScript Biotech Corporation using a solid-phase synthesis method.

| No. | Sequences |
|---|---|
| AMP29-V1 | AKIFKRIVKIIKRLL (SEQ ID NO: 5) |
| AMP29-V2 | KAIFKRIVKIIKRLL (SEQ ID NO: 6) |
| AMP29-V3 | KKAFKRIVKIIKRLL (SEQ ID NO: 7) |
| AMP29-V4 | KKIAKRIVKIIKRLL (SEQ ID NO: 8) |
| AMP29-V5 | KKIFARIVKIIKRLL (SEQ ID NO: 9) |
| AMP29-V6 | KKIFKAIVKIIKRLL (SEQ ID NO: 10) |
| AMP29-V7 | KKIFKRAVKIIKRLL (SEQ ID NO: 11) |
| AMP29-V8 | KKIFKRIAKIIKRLL (SEQ ID NO: 12) |
| AMP29-V9 | KKIFKRIVAIIKRLL (SEQ ID NO: 13) |
| AMP29-V10 | KKIFKRIVKAIKRLL (SEQ ID NO: 14) |
| AMP29-V11 | KKIFKRIVKIAKRLL (SEQ ID NO: 15) |
| AMP29-V12 | KKIFKRIVKIIARLL (SEQ ID NO: 16) |
| AMP29-V13 | KKIFKRIVKIIKALL (SEQ ID NO: 17) |
| AMP29-V14 | KKIFKRIVKIIKRAL (SEQ ID NO: 18) |
| AMP29-V15 | KKIFKRIVKIIKRLA (SEQ ID NO: 19) |

### Example 6

Streptococcus mutans (ATCC 35668) was pre-cultured to the logarithmic phase. A total of 5 mL of the bacterial solution was centrifuged at 5,000 g for 10 minutes and then resuspended in brain-heart-infusion (BHI) broth (purchased from Qingdao HaiBo Biotechnology Co., Ltd., Item No. HBKP8297-5). The concentration of the bacterial solution was adjusted to approximately 2× 10⁵ CFU/mL using pre-configured McFarland standard tubes.

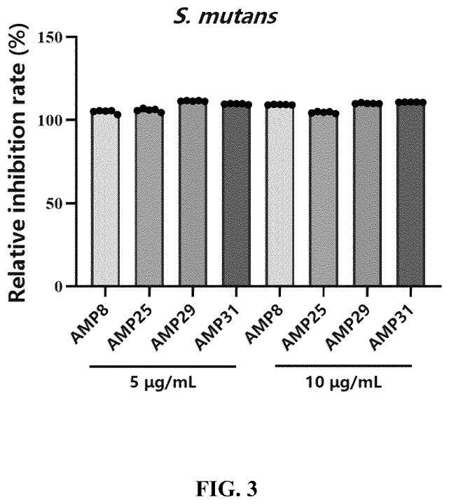

Streptococcus mutans was cultured in a 96-well microplate. To each well, 50 μL of peptide solutions prepared in Examples 1-4 at varying concentrations was added, followed by 100 μL of diluted S. mutans suspension and then 50 μL of BHI broth to achieve a final bacterial concentration of 1×10⁵ CFU/mL. The blank control contained equivalent volumes of medium and bacterial suspension without peptides, with three replicates per group. The microplate was incubated at 37 °C in a bacterial incubator with shaking at 150 rpm for 48 h. The bacterial density was expressed by OD₆₀₀, and the inhibition rate was calculated as follows: Inhibition rate (%)=((OD₆₀₀ of control bacterial solution−OD₆₀₀ of treated bacterial solution)/OD₆₀₀ of control bacterial solution)×100%. The results are shown in FIG. 3.

As shown in FIG. 3, it can be seen that after 48 hours of incubation with the addition of 5 μg/mL and 10 μg/mL of antimicrobial peptides, the antimicrobial peptide AMP8 prepared in Example 1, the antimicrobial peptide AMP25 prepared in Example 2, the antimicrobial peptide AMP29 prepared in Example 3, and the antimicrobial peptide AMP31 prepared in Example 4 inhibited Streptococcus mutans by 100%.

Conclusion: the antimicrobial peptide AMP8 prepared in Example 1, the antimicrobial peptide AMP25 prepared in Example 2, the antimicrobial peptide AMP29 prepared in Example 3, and the antimicrobial peptide AMP31 prepared in Example 4 exhibited an inhibitory effect on Streptococcus mutans.

### Example 7

Porphyromonas gingivalis was pre-cultured to the logarithmic phase. A total of 5 mL of the bacterial solution was centrifuged at 5,000 g for 10 minutes and then resuspended in Columbia blood agar (purchased from Qingdao HaiBo Biotechnology Co., Ltd., Item No. HB0124). The concentration of the bacterial solution was adjusted to approximately 2×10⁵ CFU/mL by using pre-configured McFarland standard tubes.

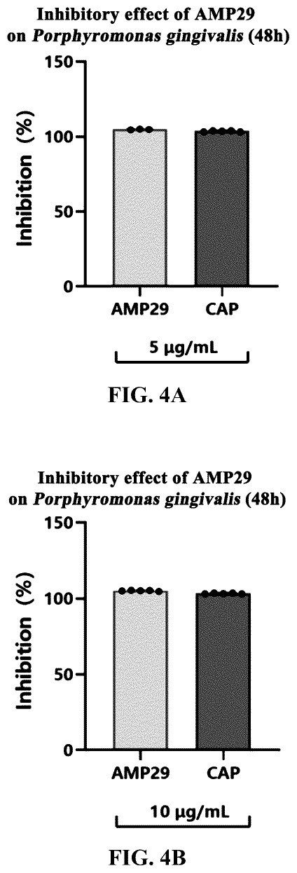

Porphyromonas gingivalis was cultured in a 96-well microplate. To each well, 50 μL of different concentrations of the antimicrobial peptide AMP29 solution prepared in Example 3 was added, followed by 100 μL of diluted Porphyromonas gingivalis suspension and then 50 μL of Columbia blood agar to achieve a final bacterial concentration of 1×10⁵ CFU/mL. The blank control contained equivalent volumes of medium and bacterial suspension without peptides, with three replicates per group. The microplate was incubated at 37 °C in a bacterial incubator with shaking at 150 rpm for 48 h. The bacterial density was expressed by OD₆₀₀, and the inhibition rate was calculated as follows: Inhibition rate (%)=((OD₆₀₀ of control bacterial solution−OD₆₀₀ of treated bacterial solution)/OD₆₀₀ of control bacterial solution)×100%. The results are shown in FIGS. 4A-4B.

As shown in FIGS. 4A-4B, it can be seen that after 48 hours of incubation with the addition of 5 μg/mL and 10 μg/mL of antimicrobial peptides, the antimicrobial peptide AMP29 prepared in Example 3 inhibited Porphyromonas gingivalis by 100%.

Conclusion: the antimicrobial peptide AMP29 prepared in Example 3 has an inhibitory effect on Porphyromonas gingivalis.

### Example 8

The example relates to liquid culture antibacterial inhibition assay. Helicobacter Pylori was pre-cultured to the logarithmic phase. A total of 4.5 mL of the bacterial solution was centrifuged at 5,000 g for 6 minutes and then resuspended in Wilkins-Chalgren medium (purchased from Qingdao Rishui Biotechnology Co., Ltd., Item No. 11071). The concentration of the bacterial solution was adjusted to approximately 2×10⁵ CFU/mL by using pre-configured McFarland standard tubes.

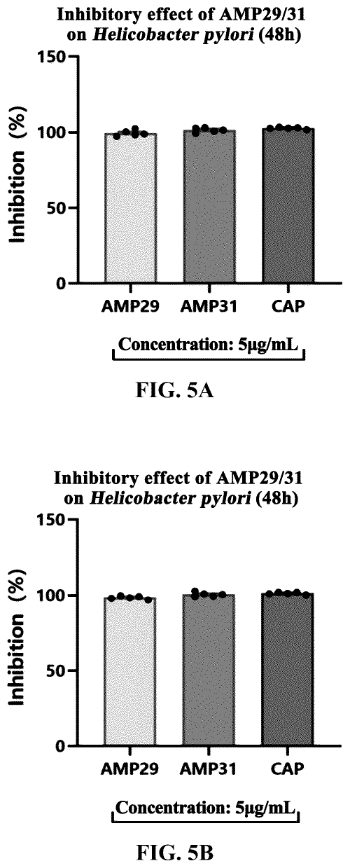

Helicobacter Pylori was cultured in a 96-well microplate. To each well, 50 μL of different concentrations of the antimicrobial peptide AMP29 solution prepared in Example 3 and the antimicrobial peptide AMP31 solution prepared in Example 4 was added, followed by 100 μL of diluted Helicobacter Pylori suspension and then 50 μL of the medium to achieve a final bacterial concentration of 1×10⁵ CFU/mL. The blank control contained equivalent volumes of medium and bacterial suspension without peptides, with three replicates per group. The microplate was incubated at 37 °C in a bacterial incubator with shaking at 150 rpm for 48 h. The bacterial density was expressed by OD₆₀₀, and the inhibition rate was calculated as follows: Inhibition rate (%)=((OD₆₀₀ of control bacterial solution−OD₆₀₀ of treated bacterial solution)/OD₆₀₀ of control bacterial solution)×100%. The results are shown in FIGS. 5A-5B.

As shown in FIGS. 5A-5B, it can be seen that after 48 hours of incubation with the addition of 5 μg/mL and 10 μg/mL of antimicrobial peptides, the antimicrobial peptide AMP29 prepared in Example 3 and the antimicrobial peptide AMP31 prepared in Example 4 inhibited Helicobacter Pylori by 100%.

Conclusion: the antimicrobial peptide AMP29 prepared in Example 3 and the antimicrobial peptide AMP31 prepared in Example 4 exhibited an inhibitory effect on Helicobacter Pylori.

### Example 9

(1) Candida albicans was inoculated into YM liquid medium (Beijing Solarbio Science and Technology, LA6970) and cultured overnight under aerobic conditions at 28 °C and 1800 rpm on a shaking incubator.
(2) A total of 200 μL of YM liquid culture medium and a predetermined concentration of a peptide was added to a 96-deep-well plate. Then, 10 μL of the Candida albicans solution was inoculated (mixed well before inoculation).
(3) After the bacteria were incubated at 1800 rpm for 48 hours on the shaking incubator at 28 °C, the turbidity of the bacterial solution was observed. A total of 100 μL of the solution (after mixing thoroughly by repeated pipetting) was taken, and the OD at 630 nm was measured using an enzyme-linked immunosorbent assay (ELISA) reader.
(4) Calculate the Percentage of Bacterial Viability: % Viability=(OD measurement−ODneg)/(ODpos−ODneg)*100

OD Measurement: OD value of the test peptide.

ODneg: OD value with no bacterial inoculation, containing only LB medium.

ODpos: OD value with bacterial inoculation and without inhibitors.

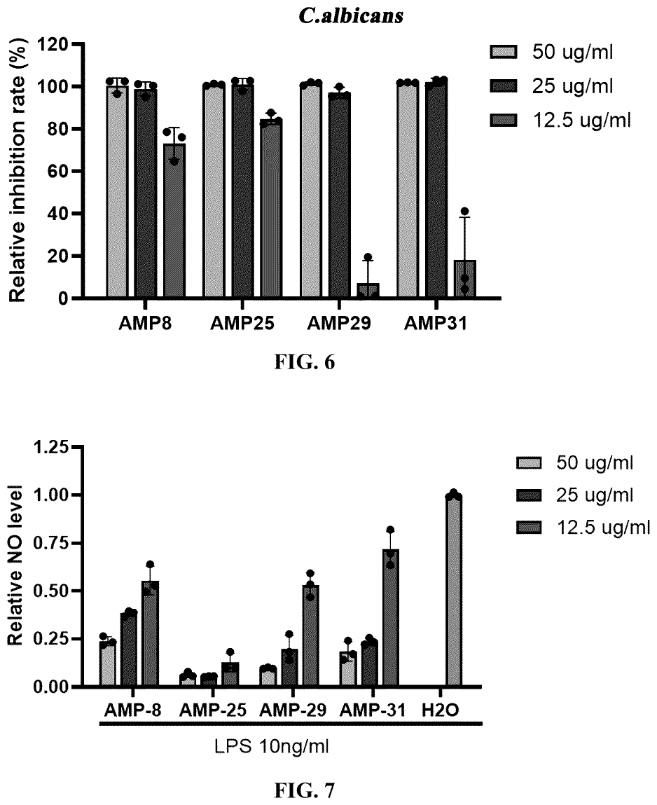

As shown in FIG. 6, it can be seen that after 48 hours of incubation with the addition of 50 μg/mL and 25 g/mL of antimicrobial peptides, the antimicrobial peptide AMP8 prepared in Example 1, the antimicrobial peptide AMP25 prepared in Example 2, the antimicrobial peptide AMP29 prepared in Example 3, and the antimicrobial peptide AMP31 prepared in Example 4 inhibited Candida albicans by 100%.

Conclusion: the antimicrobial peptide AMP8 prepared in Example 1, the antimicrobial peptide AMP25 prepared in Example 2, the antimicrobial peptide AMP29 prepared in Example 3, and the antimicrobial peptide AMP31 prepared in Example 4 inhibited exhibited an inhibitory effect on Candida albicans.

### Example 10

1. Mouse macrophage RAW264.7 cells were cultured in DMEM supplemented with 10% fetal bovine serum (FBS) and 1% penicillin-streptomycin (P/S) at 37 °C in a 5% CO₂ incubator.
2. A total of 5×10⁴ mouse-derived macrophages RAW264.7 were cultured in 12-well cell culture plates for 24 hours. Then, 20 μL of PBS (negative control), Dexamethasone (positive control), and the test peptide was added to the wells, and the cells were cultured for an additional 2 hours. After that, 10 ng/ml of lipopolysaccharide (LPS) was added to the RAW264.7 cells, and the mixture was incubated for another 24 hours.
3. The supernatant was collected, and the NO level was measured using a Biotin NO Assay Kit (S0021S). The results are shown in FIG. 7.

As shown in FIG. 7, it can be seen that after 12 hours of incubation with the addition of 50 μg/mL, 25 μg/mL and 12.5 μg/mL of antimicrobial peptides, the antimicrobial peptide AMP8 prepared in Example 1, the antimicrobial peptide AMP25 prepared in Example 2, the antimicrobial peptide AMP29 prepared in Example 3, and the antimicrobial peptide AMP31 prepared in Example 4 inhibited the LPS-induced NO production.

### Example 11

Streptococcus mutans (ATCC 35668) was pre-cultured to the logarithmic phase. A total of 5 mL of the bacterial solution was centrifuged at 5,000 g for 10 minutes and then resuspended in brain-heart-infusion (BHI) broth (purchased from Qingdao HaiBo Biotechnology Co., Ltd., Item No. HBKP8297-5). The concentration of the bacterial solution was adjusted to approximately 2×10⁵ CFU/mL by using pre-configured McFarland standard tubes.

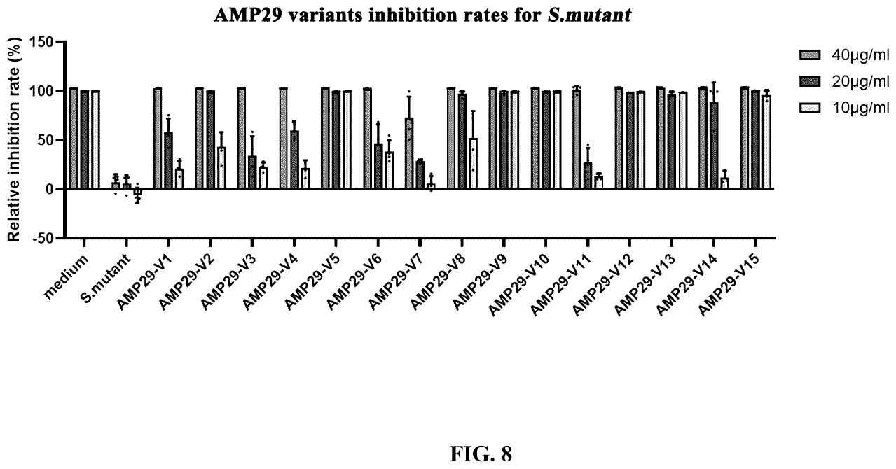

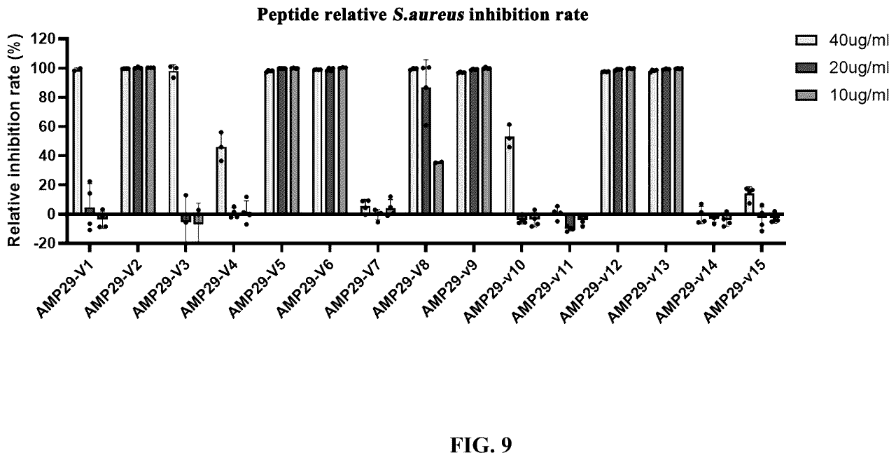

Streptococcus mutans was cultured in a 96-well microplate. To each well, 50 μL of peptide solutions prepared in Example 5 at varying concentrations was added, followed by 100 μL of diluted S. mutans suspension and then 50 μL of BHI broth to achieve a final bacterial concentration of 1×10⁵ CFU/mL. The blank control contained equivalent volumes of medium and bacterial suspension without peptides, with three replicates per group. The microplate was incubated at 37 °C in a bacterial incubator with shaking at 150 rpm for 48 h. The bacterial density was expressed by OD 600, and the inhibition rate was calculated as follows: Inhibition rate (%)=((OD₆₀₀ of control bacterial solution−OD₆₀₀ of treated bacterial solution)/OD₆₀₀ of control bacterial solution)×100%. The results are shown in FIG. 8.

As shown in FIG. 8, after 48 hours of incubation with the addition of 40, 20, and 10 μg/mL of the variants V5, V9, V10, V12, V13, and V15 of the antimicrobial peptide AMP29, AMP29 inhibited Streptococcus mutans by 100%, demonstrating a strong inhibitory effect. The motif K-K-I-F-X-R-I-V-X-X-I-X-X-L-X (SEQ ID NO: 20) is a crucial amino acid sequence responsible for maintaining the peptide's antimicrobial activity against Streptococcus mutans.

### Example 12

Staphylococcus aureus ATCC: 25923 was pre-cultured to the logarithmic phase. A total of 5 mL of the bacterial solution was centrifuged at 5,000 g for 10 minutes and then resuspended in brain-heart-infusion (BHI) broth (purchased from Qingdao HaiBo Biotechnology Co., Ltd., Item No. HBKP8297-5). The concentration of the bacterial solution was adjusted to approximately 2×10⁵ CFU/mL by using pre-configured McFarland standard tubes.

Staphylococcus aureus was cultured in a 96-well microplate. To each well, 50 μL of peptide solutions prepared in Example 5 at varying concentrations was added, followed by 100 μL of diluted Staphylococcus aureus suspension and then 50 μL of BHI broth to achieve a final bacterial concentration of 1×10⁵ CFU/mL. The blank control contained equivalent volumes of medium and bacterial suspension without peptides, with three replicates per group. The microplate was incubated at 37 °C in a bacterial incubator with shaking at 150 rpm for 48 h. The bacterial density was expressed by OD₆₀₀, and the inhibition rate was calculated as follows: Inhibition rate (%)=((OD₆₀₀ of control bacterial solution−OD₆₀₀ of treated bacterial solution)/OD₆₀₀ of control bacterial solution)×100%. The results are shown in FIG. 9.

As shown in FIG. 9, after 48 hours of incubation with the addition of 40, 20, and 10 μg/mL of the variants V2, V5, V6, V9, V12, and V13 of the antimicrobial peptide AMP29, AMP29 inhibited Staphylococcus aureus by 100%, demonstrating a significant inhibitory effect. Additionally, since the variants of AMP29 were generated via alanine scanning, these antibacterial results further demonstrate that the motif K-X-I-F-X-X-I-V-X-I-I-X-X-L-L (SEQ ID NO: 21) is a crucial amino acid sequence responsible for maintaining the peptide's antimicrobial activity against Staphylococcus aureu.

In summary, the antimicrobial peptides AMP8 prepared in Example 1, AMP25 prepared in Example 2, AMP29 prepared in Example 3, and AMP31 prepared in Example 4 all exhibit strong inhibitory effects against pathogenic microorganisms and demonstrate significant suppression of LPS-induced inflammatory responses in RAW264.7 cells.

It will be obvious to those skilled in the art that changes and modifications may be made; therefore, the aim in the appended claims is to cover all such changes and modifications.

What is claimed is:

1. An antimicrobial peptide, comprising one of the following polypeptides (1)-(7):
(1) a polypeptide comprising an amino acid sequence shown in SEQ ID NO: 1, or a polypeptide consisting of amino acid residues of SEQ ID NO: 1;
(2) a polypeptide comprising an amino acid sequence shown in SEQ ID NO: 2, or a polypeptide consisting of amino acid residues of SEQ ID NO: 2;
(3) a polypeptide comprising an amino acid sequence shown in SEQ ID NO: 3, or a polypeptide consisting of amino acid residues of SEQ ID NO: 3;
(4) a polypeptide comprising an amino acid sequence shown in SEQ ID NO: 4, or a polypeptide consisting of amino acid residues of SEQ ID NO: 4;
(5) a polypeptide resulting from substitution, deletion, and/or addition of one or more amino acid residues of the polypeptide in (1), (2), (3) or (4);
(6) a polypeptide obtained by modifying a N-terminus and/or a C-terminus of the polypeptide in (1), (2), (3) or (4); and
(7) a polypeptide having at least 85%, 90%, 95%, 98%, or 99% sequence identity to the amino acid sequence of the polypeptide in (1), (2), (3) or (4).

2. The antimicrobial peptide of claim 1, wherein the antimicrobial peptide is antimicrobial peptide AMP8, antimicrobial peptide AMP25, antimicrobial peptide AMP29 or antimicrobial peptide AMP31.

3. The antimicrobial peptide of claim 2, wherein the amino acid sequence of the antimicrobial peptide AMP8 is shown in SEQ ID NO: 1, the amino acid sequence of the antimicrobial peptide AMP25 is shown in SEQ ID NO: 2, the amino acid sequence of the antimicrobial peptide AMP29 is shown in SEQ ID NO: 3, and the amino acid sequence of the antimicrobial peptide AMP31 is shown in SEQ ID NO: 4.

4. The antimicrobial peptide of claim 3, wherein the amino acid sequence of the antimicrobial peptide AMP29 has a motif K-K-I-F-X-R-I-V-X-X-I-X-X-L-X.

5. The antimicrobial peptide of claim 3, wherein the amino acid sequence of the antimicrobial peptide AMP29 has a motif K-X-I-F-X-X-I-V-X-I-I-X-X-L-L.

6. A method for preparing the antimicrobial peptide of claim 1, the method being a solid-phase synthesis method.

7. An expression gene for expressing the antimicrobial peptide of claim 1 for maintaining a balance of oral flora.

8. The expression gene of claim 7, wherein the expression gene is configured to express the antimicrobial peptide AMP8, the antimicrobial peptide AMP25, the antimicrobial peptide AMP29, or the antimicrobial peptide AMP31.

9. The expression gene of claim 8, wherein an amino acid sequence of the antimicrobial peptide AMP8 is shown in SEQ ID NO: 1; an amino acid sequence of the antimicrobial peptide AMP25 is shown in SEQ ID NO: 2; an amino acid sequence of the antimicrobial peptide AMP29 is shown in SEQ ID NO: 3; and an amino acid sequence of the antimicrobial peptide AMP31 is shown in SEQ ID NO: 4.

10. A method for prevention or treatment of an oral disease caused by dysbiosis of oral microbiota, the method comprising: administering to a patient in need thereof a product comprising a therapeutically effective amount of an antimicrobial peptide selected from one of the following polypeptides (1)-(7):
(1) a polypeptide comprising an amino acid sequence shown in SEQ ID NO: 1, or a polypeptide consisting of amino acid residues of SEQ ID NO: 1;
(2) a polypeptide comprising an amino acid sequence shown in SEQ ID NO: 2, or a polypeptide consisting of amino acid residues of SEQ ID NO: 2;
(3) a polypeptide comprising an amino acid sequence shown in SEQ ID NO: 3, or a polypeptide consisting of amino acid residues of SEQ ID NO: 3;
(4) a polypeptide comprising an amino acid sequence shown in SEQ ID NO: 4, or a polypeptide consisting of amino acid residues of SEQ ID NO: 4;
(5) a polypeptide resulting from substitution, deletion, and/or addition of one or more amino acid residues of the polypeptide in (1), (2), (3) or (4);
(6) a polypeptide obtained by modifying a N-terminus and/or a C-terminus of the polypeptide in (1), (2), (3) or (4); and
(7) a polypeptide having at least 85%, 90%, 95%, 98%, or 99% sequence identity to the amino acid sequence of the polypeptide in (1), (2), (3) or (4).

11. The method of claim 10, wherein the oral disease is selected from the group consisting of pulpitis, apical periodontitis, gingivitis, periodontitis, stomatitis, and dental caries.

12. The method of claim 10, wherein the product is selected from the group consisting of toothpaste, mouthwash, effervescent mouthwash tablets, oral care products, and topical medicaments.


SEQUENCE LISTING (21 sequences)

SEQ ID NO: 1 - protein, 14 aa, synthetic construct
KLVKRVLKKLRKLF

SEQ ID NO: 2 - protein, 15 aa, synthetic construct
AKRIVKLIKNFFRKL

SEQ ID NO: 3 - protein, 15 aa, synthetic construct
KKIFKRIVKIIKRLL

SEQ ID NO: 4 - protein, 14 aa, synthetic construct
KILGKLLKWASKIW

SEQ ID NO: 5 - protein, 15 aa, synthetic construct
AKIFKRIVKIIKRLL

SEQ ID NO: 6 - protein, 15 aa, synthetic construct
KAIFKRIVKIIKRLL

SEQ ID NO: 7 - protein, 15 aa, synthetic construct
KKAFKRIVKIIKRLL

SEQ ID NO: 8 - protein, 15 aa, synthetic construct
KKIAKRIVKIIKRLL

SEQ ID NO: 9 - protein, 15 aa, synthetic construct
KKIFARIVKIIKRLL

SEQ ID NO: 10 - protein, 15 aa, synthetic construct
KKIFKAIVKIIKRLL

SEQ ID NO: 11 - protein, 15 aa, synthetic construct
KKIFKRAVKIIKRLL

SEQ ID NO: 12 - protein, 15 aa, synthetic construct
KKIFKRIAKIIKRLL

SEQ ID NO: 13 - protein, 15 aa, synthetic construct
KKIFKRIVAIIKRLL

SEQ ID NO: 14 - protein, 15 aa, synthetic construct
KKIFKRIVKAIKRLL

SEQ ID NO: 15 - protein, 15 aa, synthetic construct
KKIFKRIVKIAKRLL

SEQ ID NO: 16 - protein, 15 aa, synthetic construct
KKIFKRIVKIIARLL

SEQ ID NO: 17 - protein, 15 aa, synthetic construct
KKIFKRIVKIIKALL

SEQ ID NO: 18 - protein, 15 aa, synthetic construct
KKIFKRIVKIIKRAL

SEQ ID NO: 19 - protein, 15 aa, synthetic construct
KKIFKRIVKIIKRLA

SEQ ID NO: 20 - protein, 15 aa, synthetic construct
KKIFXRIVXXIXXLX

SEQ ID NO: 21 - protein, 15 aa, synthetic construct
KXIFXXIVXIIXXLL